(19) United States
(12) Patent Application Publication
(10) Pub. No.: US 2025/0032573 A1
(43) Pub. Date: Jan. 30, 2025

(54) IMMUNITY-ENHANCING PEPTIDE ANALOGS AND USES THEREOF

(71) Applicants: RESEARCH & BUSINESS FOUNDATION SUNGKYUNKWAN UNIVERSITY, Suwon-si (KR); INDUSTRY-ACADEMIC COOPERATION FOUNDATION, YONSEI UNIVERSITY, Seoul (KR)

(72) Inventors: Yoe Sik BAE, Seongnam-si (KR); Nam Joo YUN, Suwon-si (KR); Hyeon Jin CHO, Incheon (KR); Soo Hyuk CHOI, Seoul (KR); Hye Rim YOON, Seoul (KR); Hee Won CHOI, Seoul (KR); Eun Jin SEO, Seoul (KR)

(73) Assignees: RESEARCH & BUSINESS FOUNDATION SUNGKYUNKWAN UNIVERSITY, Suwon-si (KR); INDUSTRY-ACADEMIC COOPERATION FOUNDATION, YONSEI UNIVERSITY, Seoul (KR)

(21) Appl. No.: 18/627,593

(22) Filed: Apr. 5, 2024

(30) Foreign Application Priority Data
Feb. 7, 2023 (KR) 10-2023-0016048
Jun. 23, 2023 (KR) 10-2023-0081010

(51) Int. Cl. A61K 38/08; C07K 7/02

(52) U.S. Cl. CPC A61K 38/08; C07K 7/02

(57) ABSTRACT

The present invention relates to a WKYMVm peptide analog comprising an amino acid sequence of Trp (W)-Lys (K)-Tyr (Y)-Met (M)-Val (V)-D-Met (m), in which at least one amino acid residue selected from the group consisting of Lys (K), Tyr (Y), and Met (M) in the amino acid sequence is substituted with a β³-amino acid residue or a peptoid residue, and a use thereof. The WKYMVm peptide analogs of the present invention has effects of increasing stability by increasing in vivo degradation half-life and of promoting activation of innate immune responses by neutrophils through activation of formyl peptide receptors, and thus can be effectively used in preventing or treating a disease that is be prevented or treated by formyl peptide receptors, especially an infectious disease or inflammatory disease.

### CROSS-REFERENCE TO RELATED APPLICATION

This application is based on and claims priority from Korean Patent Application No. 10-2023-0016048 filed on Feb. 7, 2023; and Korean Patent Application No. 10-2023-0081010 filed on Jun. 23, 2023 in the Korean Intellectual Property Office, the content of which is incorporated herein by reference in there entireties.

### BACKGROUND

### 1. Field of the Invention

The present invention relates to a WKYMVm peptide analog; a composition comprising the analog; and an immunity-enhancing method, a method of preventing or treating an infectious disease, and a method of preventing or treating an inflammatory disease.

### 2. Related Art

Formyl peptide receptors (FPR) belong to G protein-coupled receptors involved in chemotaxis found in phagocytic cells such as neutrophils, monocytes, macrophages, and dendritic cells. It is known that humans have three types of FPRs, named as FPR1, FPR2, and FPR3. FPRs were identified with their binding ability to N-formyl peptides such as N-formylmethionine produced in bacterial or host mitochondria. Therefore, FPRs are known to be involved in mediating immune cell responses to infection.

Various substances capable of inducing the activation of FPRs in neutrophils have been developed, and among them, the synthetic peptide WKYMVm (Trp-Lys-Tyr-Met-Val-D-Met) is a powerful FPR agonist that effectively induces various innate immune responses by neutrophils through FPR activation. In particular, the WKYMVm peptide can control infectious bacteria by promoting neutrophil mobility, the production of reactive oxygen species, and degranulation activity through FPR activation. However, although the peptide WKYMVm, consisting of six residues, exhibits excellent immune activity, it has the problem of requiring continuous administration in large doses to be used as a therapeutic agent due to the problem of being rapidly decomposed in vivo.

Accordingly, the present inventors completed the present invention by developing a WKYMVm peptide analog that has increased stability in vivo and induces or promotes neutrophil activity through FPR activation.

### PRIOR ART DOCUMENTS

### Patent Document

(Patent Document 1) Korean Patent Publication No. 10-2010-0091991.

### SUMMARY

An object of the present invention is to provide a WKYMVm peptide analog comprising an amino acid sequence of Trp (W)-Lys (K)-Tyr (Y)-Met (M)-Val (V)-D-Met (m), in which at least one amino acid residue selected from the group consisting of Lys (K), Tyr (Y), and Met (M) in the amino acid sequence is substituted with a β³-amino acid residue or a peptoid residue.

Another object of the present invention is to provide a composition comprising the WKYMVm peptide analog.

Still another object of the present invention is to provide an immunity-enhancing method comprising administering the composition in a subject in need thereof.

Yet another object of the present invention is to provide a method of preventing or treating an infectious disease, comprising administering the composition in a subject in need thereof.

Yet another object of the present invention is to provide a method of preventing or treating an inflammatory disease, comprising administering the composition in a subject in need thereof.

To achieve the above objects, the present invention provides a WKYMVm peptide analog comprising an amino acid sequence of Trp (W)-Lys (K)-Tyr (Y)-Met (M)-Val (V)-D-Met (m), in which at least one amino acid residue selected from the group consisting of Lys (K), Tyr (Y), and Met (M) in the amino acid sequence is substituted with a β³-amino acid residue or a peptoid residue.

In addition, the present invention provides a composition comprising the WKYMVm peptide analog.

In addition, the present invention provides an immunity-enhancing method comprising administering the composition in a subject in need thereof.

In addition, the present invention provides a method of preventing or treating an infectious disease, comprising administering the composition in a subject in need thereof.

In addition, the present invention provides a method of preventing or treating an inflammatory disease, comprising administering the composition in a subject in need thereof.

### BRIEF DESCRIPTION OF THE DRAWINGS

FIG. 1 shows a solid-phase peptide synthesis method for the synthesis of WKYMVm peptide analogs comprising a non-natural β³-amino acid residue.

FIG. 2 shows the results of exhibiting an effect of increasing calcium ions mediated by FPR1 activation after treating an RBL-2H3 cell line with WKYMVm peptide analogs comprising a β-amino acid residue.

FIG. 3 shows the results of exhibiting an effect of increasing calcium ions mediated by FPR2 activation after treating an RBL-2H3 cell line with WKYMVm peptide analogs comprising a β-amino acid residue.

FIG. 4 shows the results of exhibiting an effect of regulating intracellular calcium ions through FPR1 and FPR2 activation by a WK_{β}Y_{β}MVm peptide analog.

FIG. 5 shows the results of exhibiting an effect of increasing calcium ions in neutrophils by WKYMVm peptide analogs comprising β-amino acid residues.

FIG. 6 shows the results of exhibiting an effect of regulating superoxide anion production in neutrophils by WKYMVm peptide analogs comprising one β-amino acid residue.

FIG. 7 shows the results of exhibiting an effect of regulating superoxide anion production in neutrophils by a WKYMVm peptide analog comprising two or three β-amino acid residues.

FIG. 8 shows the results of exhibiting an effect of activating neutrophil degranulation by WKYMVm peptide analogs comprising β-amino acid residues.

FIG. 9 shows the results of exhibiting an effect of regulating neutrophil mobility by WKYMVm peptide analogs comprising β-amino acid residues.

FIG. 10 shows the results of exhibiting an effect of regulating cytokine production in neutrophils by WKYMVm peptide analogs comprising β-amino acid residue.

FIG. 11 shows the results of exhibiting an effect of regulating cytokine production by lipopolysaccharide (LPS) in neutrophils by WKYMVm peptide analogs comprising β-amino acid residues.

FIG. 12 shows the results of exhibiting an effect of regulating neutrophil mobility by WKYMVm peptide analogs comprising peptoid residues.

FIG. 13 shows the results of exhibiting an effect of regulating production of reactive oxygen species (ROS) in neutrophils by WKYMVm peptide analogs comprising a peptoid residue.

FIG. 14 shows the results of exhibiting an effect of degranulating neutrophils by WKYMVm peptide analogs comprising peptoid residues.

FIG. 15 shows the results of measuring the degradation half-life of a WKYMVm peptide using high performance liquid chromatography (HPLC).

FIG. 16 shows the results of measuring the degradation half-life of WKYMVm peptide analogs comprising β-amino acid residues.

FIG. 17 shows the results of measuring the degradation half-life of WKYMVm peptide analogs comprising peptoid residues.

### DETAILED DESCRIPTION

Hereinafter, the present invention will be described in detail.

The terms used in the present invention have been selected as widely used general terms as possible in consideration of the functions in the present invention, but they may vary according to the intention or precedent of those skilled in the art, the emergence of new technologies and the like. In addition, in certain cases, there is also a term arbitrarily selected by the applicant, in which case the meaning will be described in detail in the detailed description of the invention. Therefore, the terms used in the present invention should be defined based on the meanings of the terms and the contents throughout the present invention, rather than the names of the simple terms.

In the present invention, when it is described to “comprise” a component or a step, this does not exclude other components or steps unless specifically stated otherwise, but it means that other components or steps may be further comprised.

The present invention provides a WKYMVm peptide analog comprising an amino acid sequence of Trp (W)-Lys (K)-Tyr (Y)-Met (M)-Val (V)-D-Met (m), in which at least one amino acid residue selected from the group consisting of Lys (K), Tyr (Y), and Met (M) in the amino acid sequence is substituted with a β³-amino acid residue or a peptoid residue.

In the present invention, one amino acid residue selected from the group consisting of Lys (K), Tyr (Y), and Met (M) in the amino acid sequence may be substituted with a β³-amino acid residue or a peptoid residue. In addition, two amino acid residues selected from the group consisting of Lys (K), Tyr (Y), and Met (M) in the amino acid sequence may be substituted with a β³-amino acid residue or a peptoid residue, and amino acid residues of Lys (K), Tyr (Y), and Met (M) in the amino acid sequence may be substituted with a β³-amino acid residue or a peptoid residue.

In the present invention, the WKYMVm peptide analog in which at least one amino acid residue selected from the group consisting of Lys (K), Tyr (Y), and Met (M) in the amino acid sequence is substituted with a β³-amino acid residue may have a following chemical formula:

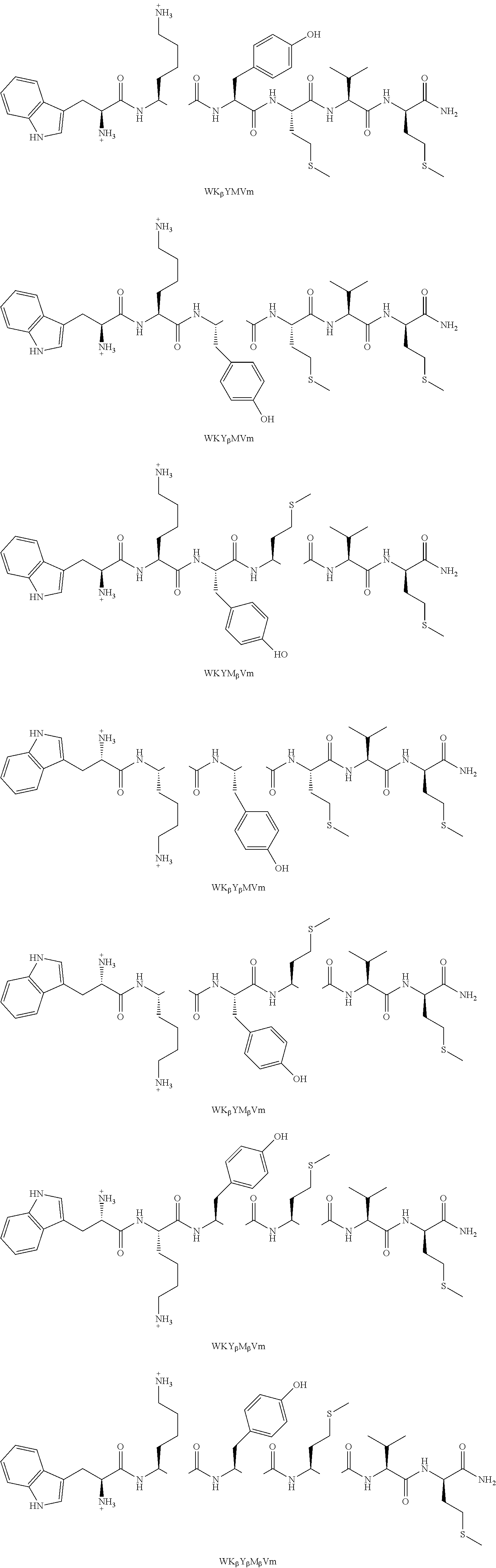

In the present invention, the WKYMVm peptide analog in which at least one amino acid residue selected from the group consisting of Lys (K), Tyr (Y), and Met (M) in the amino acid sequence is substituted with a peptoid residue have a following chemical formula:

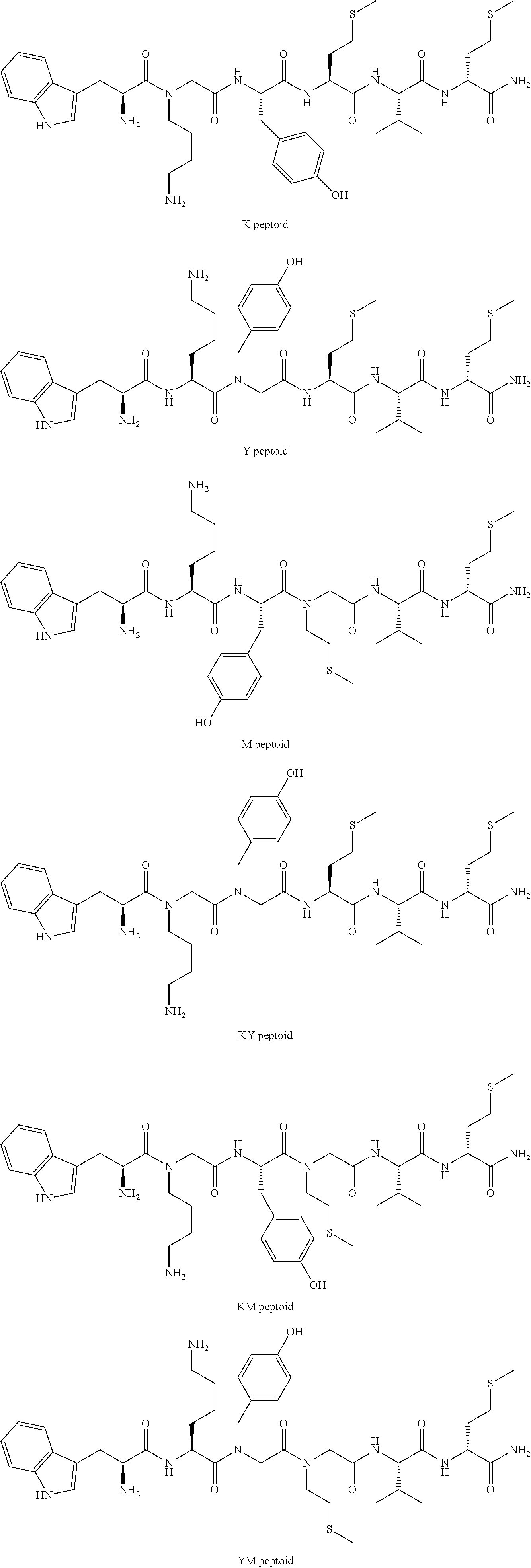

In the present invention, the WKYMVm peptide analog may induce or promote activation of a formyl peptide receptor (FPR).

As used herein, the term “FPR” belongs to G protein-coupled receptors involved in chemotaxis and a type of receptor involved in mediating immune cell responses to infection.

The WKYMVm peptide analog of the present invention was confirmed to have an effect of inducing or promoting activation of FPR1 and FPR2, similar to a WKYMVm peptide that is not substituted with a non-natural amino acid (see Example 2).

In addition, the WKYMVm peptide analog of the present invention was confirmed to have one or more characteristics selected from the group consisting of: (i) promoting activation of innate immune responses by neutrophils; (ii) inducing an increase in calcium ions in neutrophils; (iii) promoting production of reactive oxygen species (ROS) by neutrophils; (iv) promoting degranulation activity of neutrophils; (v) promoting mobility of neutrophils; and (vi) regulating cytokine production in neutrophils (see Examples 3 and 4).

In addition, the WKYMVm peptide analog was confirmed to have an increased in vivo degradation half-life compared to a WKYMVm peptide and thus the stability was significantly improved (see Example 5).

In addition, the present invention provides a composition comprising the WKYMVm peptide analog. Preferably, the composition may be a pharmaceutical composition.

The pharmaceutical composition of the present invention may further comprise a pharmaceutically acceptable carrier. As used herein, the term “pharmaceutically acceptable” means that the carrier exhibits non-toxic properties to cells or humans exposed to the composition. The carrier may be used without limitation as long as it is known in the art, such as buffers, preservatives, analgesic agents, solubilizers, isotonic agents, stabilizers, bases, excipients, lubricants, and the like.

In addition, the pharmaceutical composition of the present invention may be formulated and used in the form of oral dosage forms such as powder, granules, tablets, capsules, suspensions, emulsions, syrups, and aerosols, external preparations, suppositories, and sterile injection solutions each according to the conventional methods. Furthermore, it may be used in the form of ointments, lotions, sprays, patches, creams, powder, suspensions, gels, or gel-type external skin preparations. Examples of carriers, excipients, and diluents that may be comprised in the composition of the present invention are lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oils. When formulated, it is prepared using diluents or excipients such as commonly used fillers, extenders, binders, wetting agents, disintegrants, and surfactants.

Solid preparations for oral administration comprise tablets, pills, powder, granules, and capsules, and these solid preparations are formulated by mixing at least one excipient, for example, starch, calcium carbonate, sucrose or lactose and gelatin. Furthermore, in addition to simple excipients, lubricants such as magnesium stearate talc are also used. Liquid preparations for oral administration comprise suspensions, oral solutions, emulsions, and syrups. In addition to commonly used simple diluents such as water and liquid paraffin, various excipients such as wetting agents, sweeteners, fragrances, and preservatives may be comprised. Preparations for parenteral administration comprise sterilized aqueous solutions, non-aqueous solutions, suspensions, emulsions, freeze-dried preparations, and suppositories. As non-aqueous solvents and suspensions, propylene glycol, polyethylene glycol, vegetable oils such as olive oil, and an injectable ester such as ethyl oleate may be used. As a base for suppositories, Witepsol, Macrogol, Tween 61, cacao oil, laurel oil, glycerol, gelatin or the like may be used.

The pharmaceutical composition of the present invention is administered in a pharmaceutically effective amount. As used herein, the term “administration” means introducing a predetermined substance into an individual by an appropriate method, and the composition may be administered through any general route as long as it may reach a target tissue. It may be administered by oral administration, intraperitoneal administration, intravenous administration, intramuscular administration, subcutaneous administration, intradermal administration, topical administration, intranasal administration, intrapulmonary administration, or rectal administration, but the administration route is not limited thereto.

The term “individual” refers to all animals, comprising rats, mice, and livestock, comprising humans. Preferably, it may be a mammal, comprising humans.

The term “pharmaceutically effective amount” refers to an amount that is sufficient to treat a disease with a reasonable benefit/risk ratio applicable to medical treatment, and an effective dose level may be determined based on factors comprising the sex, age, weight, and health conditions of a patient, the type and severity of a patient's disease, drug activity, sensitivity to the drug, administration method, administration time, administration route, excretion rate, treatment duration, blending, concurrently used drugs, and other factors well known in the medical field. The recommended dose may be administered once a day, or it may be divided and administered several times.

In addition, the present invention provides an immunity-enhancing method comprising administering the composition in a subject in need thereof.

As used herein, the term “immunity-enhancing” of the present invention is one of the important therapeutic strategies that strengthens the body's defense mechanism against various diseases such as infectious diseases, inflammatory diseases, and cancer, and the activity of immune cells may be increased to stimulate immune responses and obtain an immunity-enhancing effect. For example, phagocytes play a major role in immune responses, and phagocytosis, a major role of macrophages, absorbs microorganisms and other pyrogenic particles, and also stimulates immune responses by producing cytokines such as tumor necrosis factor-α (TNF-α), IL-1β, and IL-12 and secreting cytotoxic and inflammatory substances such as nitric oxide (NO). Therefore, increasing macrophage activity may be a means to enhance immunity. Since infections and diseases mainly occur when immune function is reduced, various studies are being conducted on how to enhance these immune responses through immune-enhancing substances when the function of the body's immune system is reduced.

The immunity may be innate immunity or acquired immunity, and preferably it may mean both innate immunity and acquired immunity.

In addition, the present invention provides a method of preventing or treating an infectious disease, comprising administering the composition in a subject in need thereof.

In the present invention, the infectious disease may be selected from the group consisting of respiratory syncytial (RS) virus infection, keratitis, conjunctivitis, tuberculosis, tuberculous osteomyelitis, tuberculous peritonitis, tuberculous meningitis, cervical lymphadenitis, infectious myositis, fungal infection, acute viral hepatitis, acute gastroenteritis, acute vaginitis, Kikuchi disease, norovirus enteritis, brain abscess, encephalitis, herpes simplex, herpes simplex encephalitis, shingles, Escherichia coli infection, dengue fever, rash, tinea capitis, legionellosis, leptospirosis, listeriosis, malaria, syphilis, melioidosis, viral meningitis, bacterial meningitis, botulism, brucellosis, Vibrio vulnificus sepsis, bacterial shigellosis, Pseudomonas infection, hand, foot-and-mouth disease, amoebic dysentery, lymphadenitis, cervicitis, enterohemorrhagic Escherichia coli infection, sepsis, pulmonary tuberculosis, and staphylococcal infection, but is not limited thereto.

In addition, the present invention provides a method of preventing or treating an inflammatory disease, comprising administering the composition in a subject in need thereof.

In the present invention, the inflammatory disease may be selected from the group consisting of ankylosing spondylitis, psoriatic arthritis, osteoarthritis, rheumatoid arthritis, arthritis, dermatitis, atopic dermatitis, conjunctivitis, periodontitis, gingivitis, rhinitis, allergic rhinitis, chronic sinusitis, otitis media, pharyngitis, tonsillitis, bronchitis, pneumonia, emphysema, bronchial asthma, pulmonary fibrosis, gastric ulcer, gastritis, Crohn's disease, enteritis, colitis, hemorrhoids, urethritis, gout, rheumatic fever, lupus, fibromyalgia, tendinitis, tenosynovitis, peritendinitis, myositis, hepatitis, cystitis, nephritis, Sjogren's syndrome, multiple sclerosis, ulcers, and wounds, but is not limited thereto.

Hereinafter, the present invention will be described in more detail through examples. These examples are for illustrating the present invention in more detail, and the scope of the present invention is not limited to these examples.

### Example 1. Synthesis of Peptide Analogs in which a Part of the WKYMVm Peptide is Substituted with a Non-Natural Residue

### 1.1. Design of Non-Natural Residue-Substituted Analogs

The WKYMVm peptide consists of six residues and except for the D-methionine (m) at the C-terminus, the remaining residents are natural L-amino acids. Therefore, the WKYMVm peptide has a short in vivo half-life, making it difficult to develop into a drug. As a method to improve this, an attempt was made to increase in vivo stability by substituting some of the L-amino acid residues in the peptide with non-natural residues to delay metabolism by proteolytic enzymes.

### 1.2. Selection of Non-Natural Residues

In the present invention, a β³-amino acid, in which a methylene (—CH₂) group was added between the α-carbon and the carboxyl group of a natural L-α-amino acid, and three types of peptoid residues, in which a side chain of the α-amino acid was transferred to an amide group, were selected as non-natural residues, and a part of the WKYMVm residues were substituted with the non-natural residues (see Scheme 1).

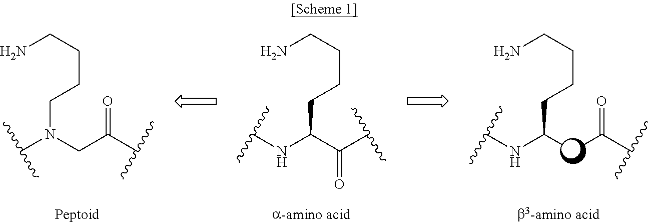 [SMILES: *C(=O)CN(*)CCCCN ; *N[C@@H](CCCCN)C(*)=O ; *N[C@@H](CCCCN)CC(*)=O]

1.3. Synthesis of WKYMVm Peptide Analogs to which a β-Amino Acid Residue has been Introduced

Eight peptide analogs were synthesized by substituting a part of the five residues among the WKYMVm residues, except for the D-methionine (m) at the C-terminus, with a non-natural β³-amino acid. The eight peptide analogs synthesized were: four peptides comprising one non-natural amino acid residue (WK_{β}YMVm, WKY_{β}MVm, WKYM_{β}Vm, WKYMV_{β}m), three peptides comprising two non-natural amino acid residues (WK_{β}Y_{β}MVm, WK_{β}YM_{β}Vm, WKY_{β}M_{β}Vm), and one peptide (WK_{β}Y_{β}M_{β}Vm) comprising three non-natural residues (group represented by Chemical Formula 1).

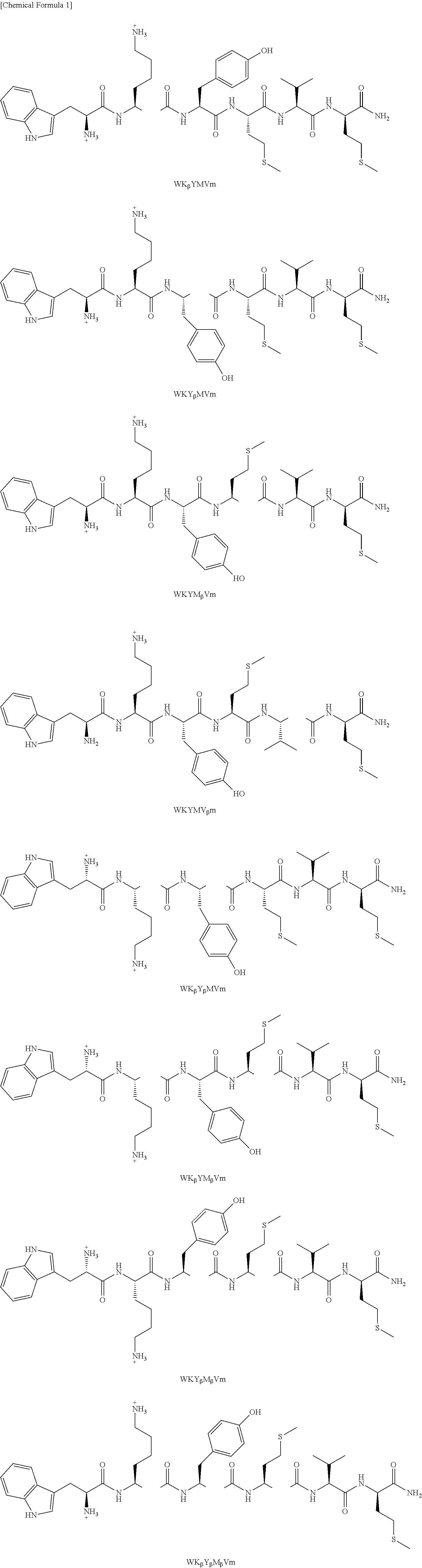

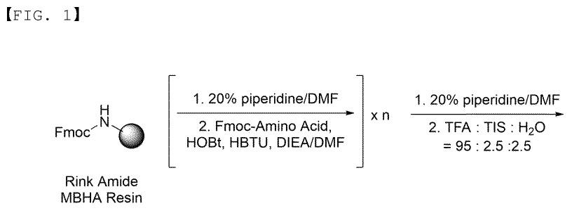

The method of synthesizing the WKYMVm peptide analogs substituted with non-natural a β³-amino acid (solid-phase synthesis method, FIG. 1) is described below.

1) A Rink Amide methylbenzhydrylamine (MBHA) resin (1.0 equivalent) and 3 to 5 mL of dimethylformamide (DMF) were added to a solid phase extraction (SPE) tube, the resin was soaked for one hour, and then the DMF was removed. 3 to 5 mL of 20% piperidine/DMF solution was added and the SPE tube was shaken for one hour. Thereafter, the solvent was removed, and the resin was washed with 3 to 5 times with 3 mL of DMF.

2) Fluorenylmethoxycarbonyl (Fmoc)-amino acid in which the N-terminus was protected with the Fomc group (3.0 equivalents), hydroxybenzotriazole (HOBt) (2.7 equivalents), and hexafluorophosphate benzotriazole tetramethyl uronium (HBTU) (2.7 equivalents) were added into a 1 M N,N-diisopropylethylamine (DIPEA)/DMF solution to activate the amino acids for 15 minutes.

3) The activated Fmoc-amino acid solution was put into an SPE tube and stirred for one to two hours: an α-amino acid was stirred for one hour and a β-amino acid was stirred for two hours. Thereafter, the resin was washed three times with DMF and twice with dichloromethane (DCM).

4) 3 to 5 mL of 20% piperidine/DMF solution was added into the SPE tube and stirred for 30 minutes to remove the Fmoc group, and then the resin was washed for 3 to 5 times with DMF.

5) Steps 2) to 4) were repeated according to the peptide sequence.

6) After preparing a final removal solution containing the composition of trifluoroacetic acid (TFA) (20%), water (2.5%), and triisopropylsilane (TIS) (2.5%), 2 to 3 mL of the solution was added to the resin and the resulting mixture was allowed to stand for three hours.

7) After filtering and removing the resin, nitrogen gas was blown to concentrate the filtrate solution to 1 mL.

8) 15 to 30 mL of cold diethyl ether was added to the filtrate solution to precipitate the peptide as a solid, and then a precipitate from the solution was separated through a centrifuge.

9) After dissolving the precipitate in dimethylsulfoxide (DMSO), the pure peptides were separated using reverse-phase high-performance liquid chromatography (HPLC) and then dried.

### 1.4. Synthesis of WKYMVm Peptide Analogs Comprising a Peptoid Residue

As an additional strategy to increase the in vivo stability of the FPR agonist, six analogs in which a part of the residues of the active peptide WKYMVm were substituted with a peptoid residue were synthesized. In the six synthesized peptide analogs, one or two of the K, Y, and M residues were substituted with a peptoid residue (N-alkylglycine). The six synthesized peptide analogs are as follows: three peptides comprising one peptoid residue (K peptoid, Y peptoid, M peptoid) and three peptides comprising two peptoid residues (KY peptoid, KM peptoid), YM peptoid) (group represented by Chemical Formula 2).

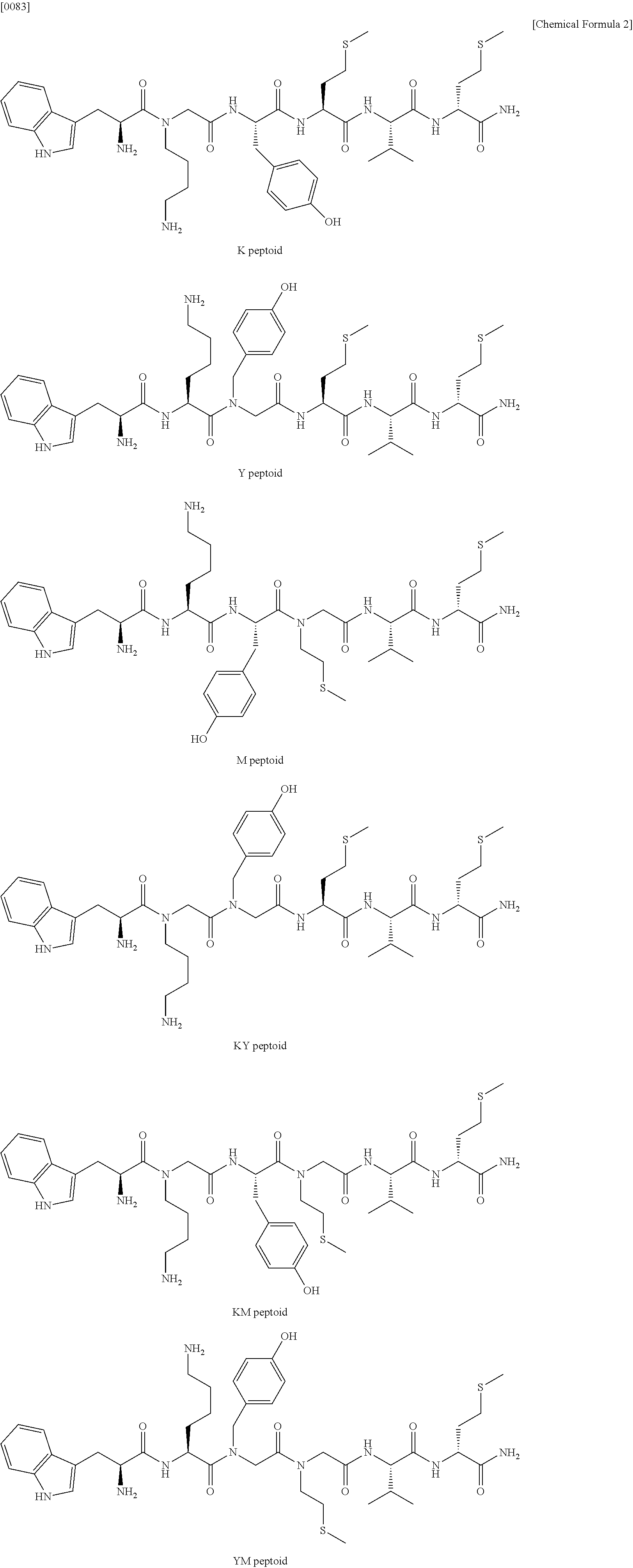

The method for synthesizing the WKYMVm peptide analogs comprising a peptoid residue was a solid-phase synthesis method, which is the method for synthesizing the WKYMVm peptide analog substituted with a β³-amino acid. In the step of introducing a peptoid residue, 2-bromoacetic acid was coupled using the N,N′-diisopropylcarbodiimide (DIC) reagent, and then the amine corresponding to the peptoid side chain was introduced through bimolecular nucleophilic substitution (S_{N}2) reaction (Scheme 2 below). In addition, 4-t-butoxybenzylamine, a reagent for substituting the tyrosine (Y) residue with a peptoid residue, was synthesized from 4-hydroxybenzonitrile and used (Scheme 3 below).

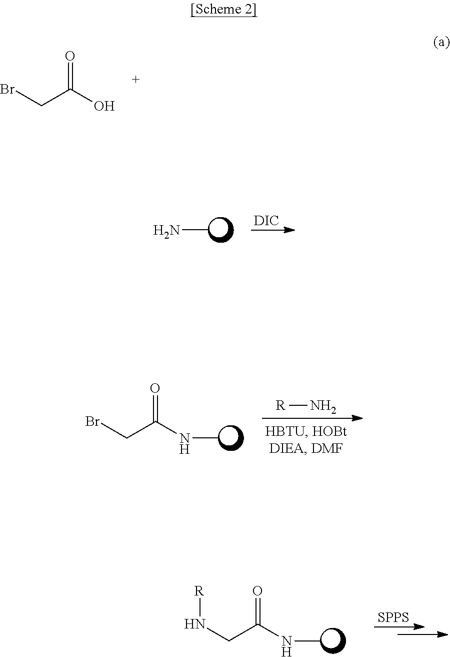

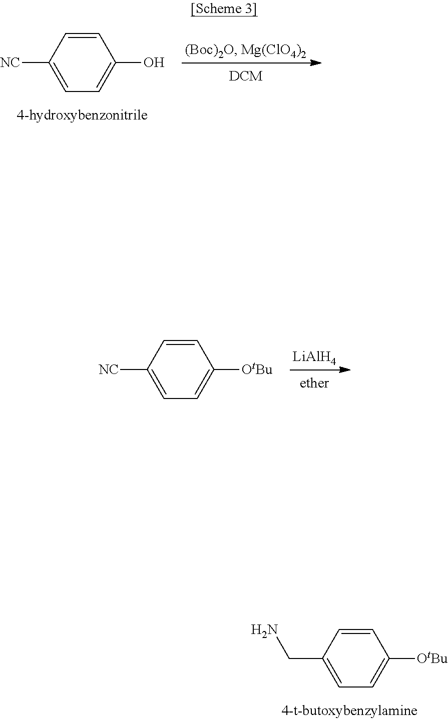 [SMILES: N#Cc1ccc(O)cc1 ; CC(C)(C)Oc1ccc(C#N)cc1 ; "LiAlH4" ; CC(C)(C)Oc1ccc(CN)cc1]

### Example 2. Measurement of FPR Activity by WKYMVm Peptide Analogs to which a β-Amino Acid Residue has been Introduced

### 2.1. FPR1 Activation by WKYMVm Peptide Analogs Containing One β-Amino Acid

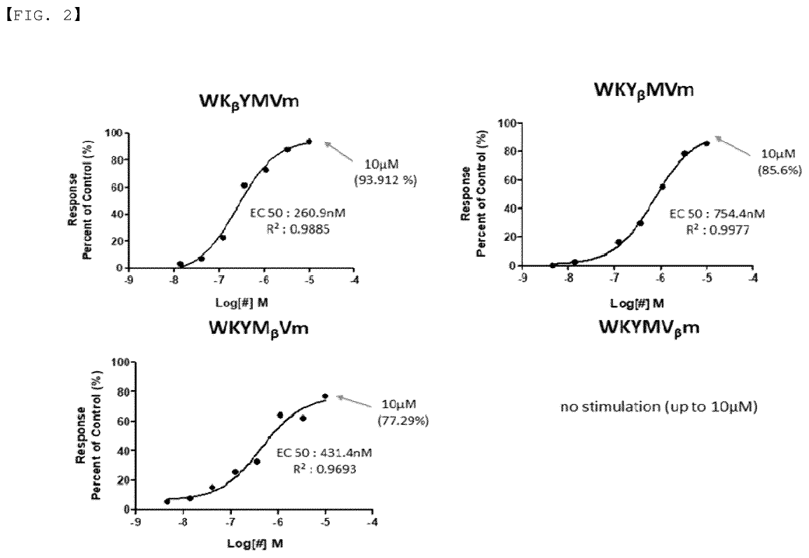

To investigate whether activation of FPR1, which is important in regulating the activity of innate immune cells, is induced by the WKYMVm peptide analogs, changes in calcium ions were measured in RBL-2H3 cell line, a type of mast cell that artificially expressed FPR1. As a result, an increase in intracellular calcium ions was induced by WK_{β}YMVm (K is a β-amino acid) through FPR1 activation in a concentration-dependent manner, and the measured EC₅₀ value was 260.9 nM (FIG. 2). An increase in calcium ions in a concentration-dependent manner was also observed in the FPR1-expressing cell line by WKY_{β}MVm (Y is a β-amino acid) and WKYM_{β}Vm (M is a β-amino acid), and the measured EC₅₀ values of these peptide analogs were 754.4 nM and 431.4 nM, respectively (FIG. 2). On the other hand, an increase in intracellular calcium ions by WKYMV_{β}m (V is a β-amino acid) through activation of FPR1 was not observed.

From the above results, it was confirmed that the induction of calcium ion-increasing activity by the WKYMVm peptides exhibited by binding of FPR1 not only to the α-amino acid residues of K, Y, and M amino acids but also to the β-amino acid residues.

### 2.2. FPR2 Activation by WKYMVm Peptide Analogs Containing One β-Amino Acid

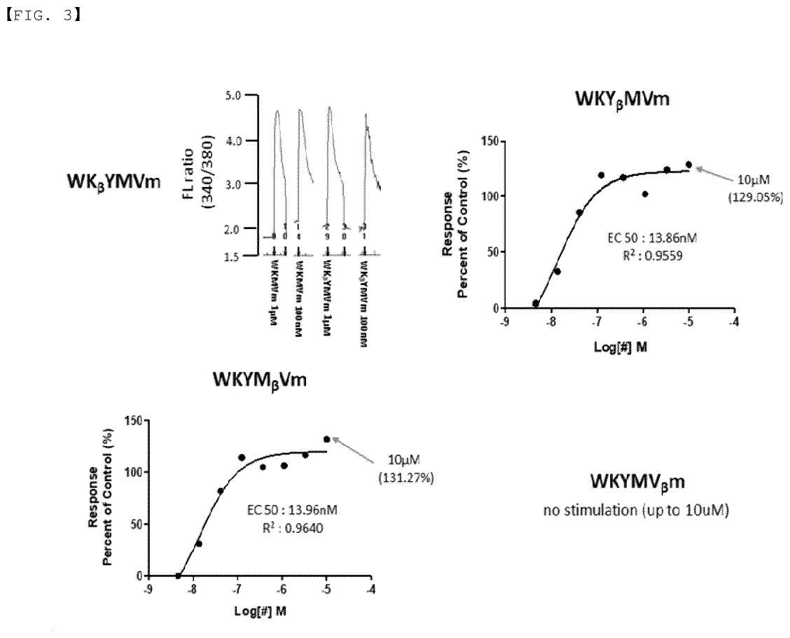

To investigate whether activation of FPR2 is induced by the WKYMVm peptide analogs, changes in calcium ions were measured in RBL-2H3 cell line that artificially expressed FPR2. As a result, an increase in calcium ions in a concentration-dependent manner was observed in the FPR2-expressing cell line by WK_{β}YMVm, WKY_{β}MVm, and WKYM_{β}Vm, and the measured EC₅₀ values of WKY_{β}MVm and WKYM_{β}Vm were 13.86 nM and 13.96 nM, respectively (FIG. 3). On the other hand, an increase in intracellular calcium ions by WKYMV_{β}m through activation of FPR2 was not observed.

### 2.3. FPR1/2 Activation by WKYMVm Peptide Analogs Containing Two β-Amino Acids

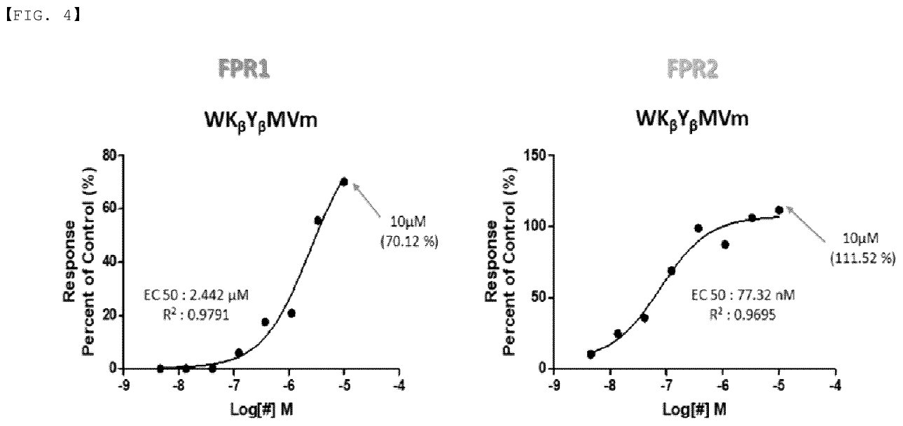

Based on the results of measuring the activity of the analogs in which one amino acid was substituted with a β-amino acid in the structure of WKYMVm, analogs in which two amino acids were simultaneously substituted with a β-amino acid was synthesized to investigate the effect of regulating intracellular calcium ions through the regulation of FPR1 and FPR2 activities. As a result, it was confirmed that FPR1 and FPR2 were each activated by WK_{β}Y_{β}MVm, including an increase in intracellular calcium ions (FIG. 4). However, the EC₅₀ values of WK_{β}Y_{β}MVm for FPR1 and FPR2 were 2.44 μM and 77.32 nM, respectively, confirming that the activity was slightly lower than that of the analogs in which one amino acid was substituted with a β-amino acid.

### Example 3. Measurement of Neutrophil Activity by WKYMVm Peptide Analogs to which a β-Amino Acid Residue was Introduced

### 3.1. Calcium Ion-Increasing Activity in Neutrophils

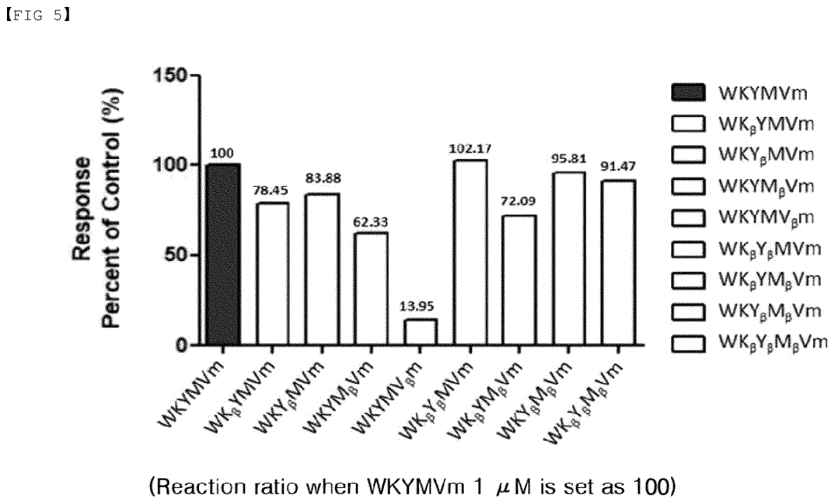

Analogs in which one, two, or three amino acids for K, Y, M, and V in the structure of WKYMVm were each replaced with a β-amino acid were synthesized, and the increase in intracellular calcium ions, which is a representative indicator of neutrophil activation, was measured to confirm the effect of these on regulating neutrophil activity. As a result, it was observed that a calcium ion-increasing effect in neutrophils was highly induced in seven peptide analogs, WK_{β}YMVm, WKY_{β}MVm, WKYM_{β}Vm, WK_{β}Y_{β}MVm, WK_{β}YM_{β}Vm, WKY_{β}M_{β}Vm, and WK_{β}Y_{β}M_{β}Vm (FIG. 5). However, a calcium ion-increasing effect in neutrophils was not exhibited in the WKYMV_{β}m peptide analog.

### 3.2. Production Activity of Reactive Oxygen Species (ROS) in Neutrophils

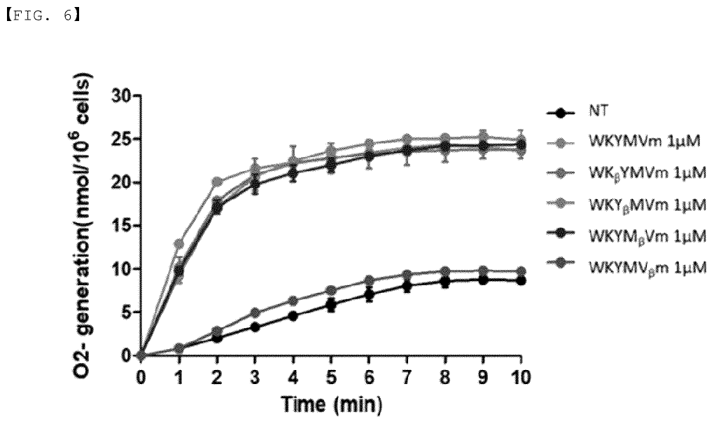

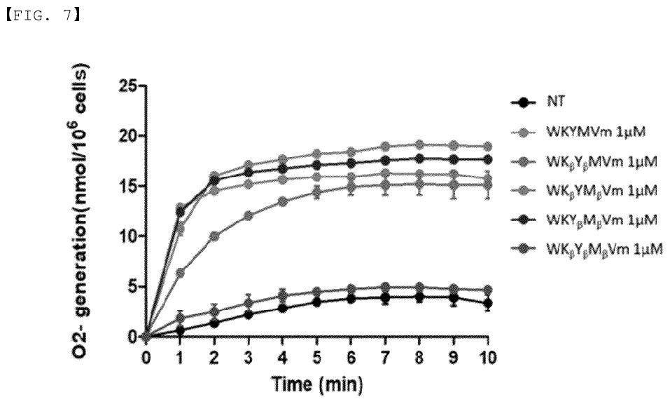

As an increase in calcium ions in neutrophils by various analogs of WKYMVm was observed, the superoxide anion production ability was measured to investigate the effect of these analogs on ROS production, which is a representative immune function of neutrophils. As a result, it was confirmed that the WK_{β}YMVm, WKY_{β}MVm, and WKYM_{β}Vm peptide analogs in which one amino acid was substituted with a β-amino acid were able to induce superoxide anion production very effectively to a similar extent as the WKYMVm peptide (FIG. 6). In addition, the WK_{β}Y_{β}MVm, WK_{β}YM_{β}Vm, and WKY_{β}M_{β}Vm peptide analogs in which two amino acids were substituted with a β-amino acid also effectively promoted superoxide anion production (FIG. 7). However, the WKYMV_{β}m and WK_{β}Y_{β}M_{β}Vm peptide analogs did not induce superoxide anion production.

### 3.3 Degranulation Activity of Neutrophils

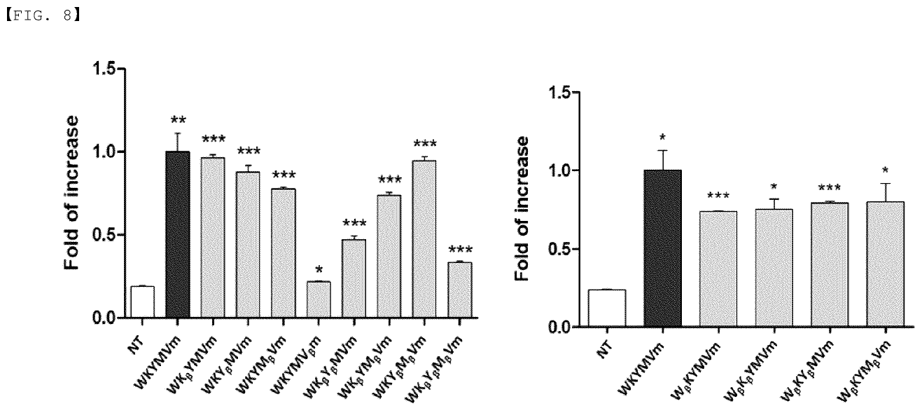

The degranulation activity of neutrophils by the analogs in which each amino acid in the structure of WKYMVm was substituted with a β-amino acid was compared and analyzed by measuring beta-hexosaminidase activity. As a result, it was confirmed that the WK_{β}YMVm, WKY_{β}MVm, and WKYM_{β}Vm peptide analogs comprising one non-natural amino acid induced the degranulation activity of neutrophils very effectively to a similar extent as the WKYMVm peptide (FIG. 8). In addition, the WK_{β}Y_{β}MVm, WK_{β}YM_{β}Vm, and WKY_{β}M_{β}Vm peptide analogs in which two amino acids were substituted with a β-amino acid also effectively promoted the degranulation activity of neutrophils (FIG. 8). However, the WKYMV_{β}m and WK_{β}Y_{β}M_{β}Vm peptide analogs did not induce the degranulation activity of neutrophils.

### 3.4. Mobility Regulating Effect of Neutrophils

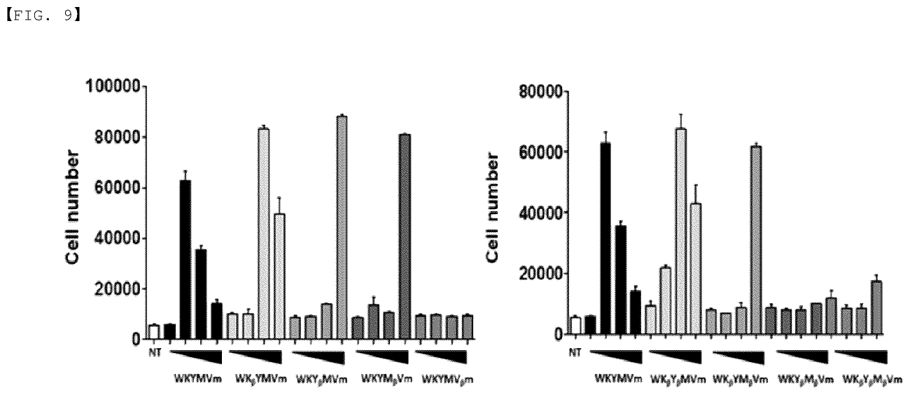

To investigate the effect of the WKYMVm peptide analogs on the regulation of immune cell mobility, which a representative function of FPR, a chemotaxis assay was performed using a Boyden chamber. As a result, it was confirmed that the WK_{β}YMVm, WKY_{β}MVm, and WKYM_{β}Vm peptide analogs in which one amino acid was substituted with a β-amino acid were able to effectively induce chemotactic migration of neutrophils in a concentration-dependent manner from a concentration of 1 nM or 10 nM to a concentration of 1 μM (FIG. 9). In particular, their neutrophil mobility showed activity similar to or slightly higher than that induced by the WKYMVm peptide at 1 μM. In the case of the WKYMV_{β}m peptide analog, neutrophil mobility was not promoted at a low concentration, but it was confirmed that a highly active neutrophil mobility promotion effect was induced at a concentration of 1 μM.

### 3.5. Cytokine Production Regulating Effect of Neutrophils

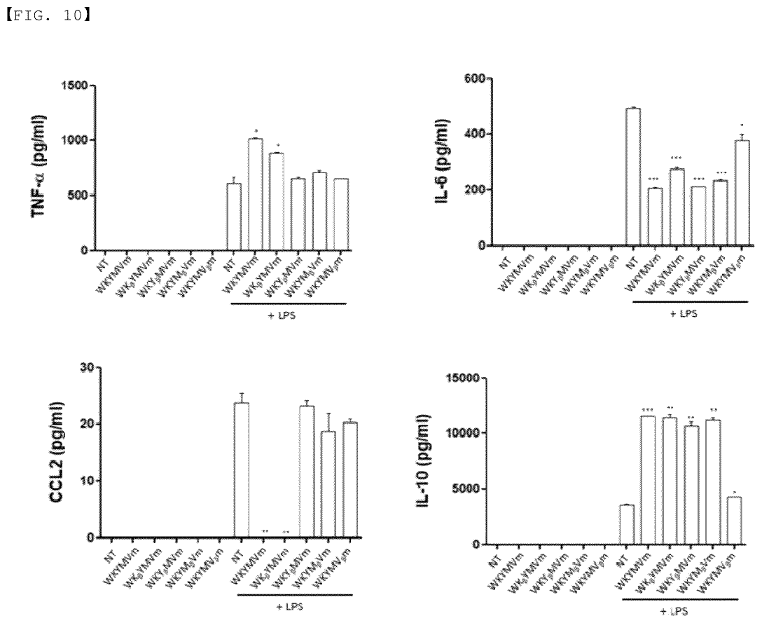

Neutrophils regulate the activity of other immune cells through the production of various types of cytokines. To investigate the effect of the WKYMVm peptide analogs on regulating cytokine production in neutrophils, neutrophils isolated from mouse bone marrow were treated with each of the WKYMVm peptide and its analogs at 10 μM, and 24 hours later, the supernatants were collected and subjected to an enzyme-linked immunosorbent assay (ELISA) to measure the amount of cytokine secretion. As a result, WKYMVm and its analogs did not induce the production of TNF-α, IL-6, CCL2, and IL-10 in neutrophils on their own. On the other hand, it was confirmed that among the cytokines produced by lipopolysaccharide (LPS) stimulation, IL-6 production was effectively inhibited by WKYMVm and its analogs, and IL-10 production was rather increased by WKYMVm and its analogs (FIG. 10).

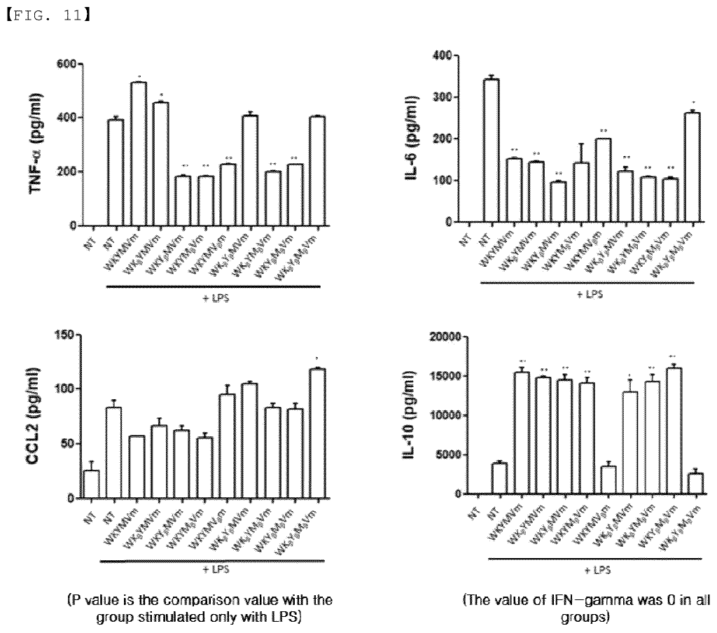

In addition, the effect of regulating the production of LPS-induced cytokines in neutrophils was confirmed after treating with WKYMVm and its analogs each at a concentration of 1 μM. As a result, it was confirmed that the WKYMVm peptide analogs effectively inhibited the production of TNF-α and IL-6 by LPS stimulation and effectively increased the production of IL-10 by LPS (FIG. 11). However, the WKYMV_{β}m peptide analog exhibited a minimal effect in regulating the neutrophil activity.

### Example 4. Measurement of Neutrophil Activity of WKYMVm Peptide Analogs Comprising a Peptoid Residue

### 4.1. Mobility Regulation by Neutrophils

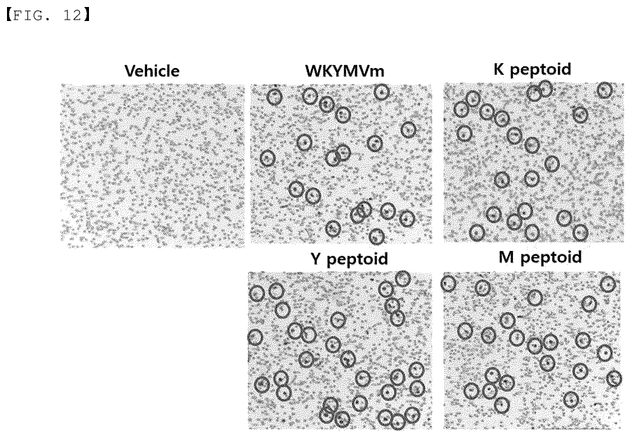

To confirm the effect of regulating neutrophil activity by peptoid-containing analogs in which a peptoid residue was substituted for K, Y, and M in the structure of WKYMVm, the effect of enhancing neutrophil mobility, which is a core function of FPR, was compared and analyzed. As a result, it was observed that the vehicle did not induce neutrophil mobility at all, but under experimental conditions where WKYMVm strongly induced neutrophil mobility, the K peptoid, Y peptoid, and M peptoid were able to promote neutrophil mobility with similar activity to the WKYMVm peptide (FIG. 12). As a result, it was confirmed that a peptoid-comprising WKYMVm peptide analog could also be developed into an FPR agonist.

### 4.2. Production of Reactive Oxygen Species (ROS) by Neutrophils

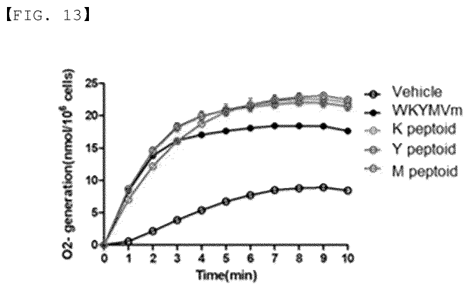

The effect of superoxide anion production in neutrophils by the peptoid-comprising analogs in which a peptoid residue was substituted for K, Y, and M in the structure of WKYMVm was measured. As a result, it was confirmed that the K peptoid, Y peptoid, and M peptoid in which a peptoid was substituted were able to effectively promote superoxide anion production (FIG. 13).

### 4.3. Degranulation Activity of Neutrophils

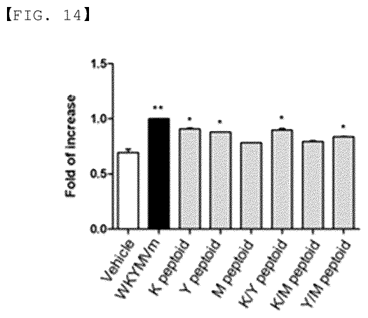

The degranulation activity of neutrophils by the peptoid-containing analogs in which a peptoid residue was substituted for K, Y, and M in the structure of WKYMVm was measured. As a result, it was confirmed that the K peptoid, Y peptoid, and M peptoid in which a peptoid was substituted promoted the degranulation activity of neutrophils to the extent similar to the WKYMVm peptide (FIG. 14).

### Example 5. Correlation Analysis of Ex Vivo Stability and In Vitro Innate Immune Activity with the Structure of the WKYMVm Peptide Analogs

5.1. Structure-Activity Correlation Analysis of WKYMVm Peptide Analogs to which a β-Amino Acid was Introduced

To measure the in vitro innate immune activity of four peptides comprising one non-natural β³-amino acid residue, the effect of enhancing neutrophil mobility was measured. As a result, the WK_{β}YMVm, WKY_{β}MVm, and WKYM_{β}Vm peptide analogs showed similar activity to the WKYMVm peptide. On the other hand, WKYMV_{β}m showed weak activity only at high concentrations. Based on these results, three peptides in which two of the three residues, K, Y, and M, excluding the two residues V and m at the C-terminus and the W residue at the N-terminus, were substituted with a non-natural residue were synthesized. Among these, the WK_{β}YM_{β}Vm and WKY_{β}M_{β}Vm peptide analogs maintained similar activity to the WKYMVm peptide, while the WKβY_{β}MVm peptide analog showed a slight decrease in activity.

From the above results, the finding that almost no immune activity was exhibited in the case of substituting valine in WKYMVm suggests that the interactions with the FPR receptors may be significantly changed by not only D-methionine at the C-terminus, which is known to be essential for inducing immune activity, but also the spatial orientation of the adjacent valine. In addition, this is consistent with the fact that the crystal structure of the FPR2 receptor, one of the FPR receptors, shows a binding form in which the C-terminal part of WKYMVm is positioned deep inside the receptor and the N-terminus is exposed to the outside of the receptor. Therefore, it was confirmed that in designing an FPR agonist analogs, it is reasonable to introduce a non-natural monomer toward the N-terminus while maintaining the two amino acid sequences (Val-D-Met) at the C-terminus. This is also in accordance with the method of linking an antifungal antimicrobial peptide (AMP) to the N-terminus of the FPR agonist when synthesizing an FPR agonist-AMP complex.

5.2. Structure-Stability Correlation Analysis of WKYMVm Peptide Analogs to which a β-Amino Acid Residue was Introduced

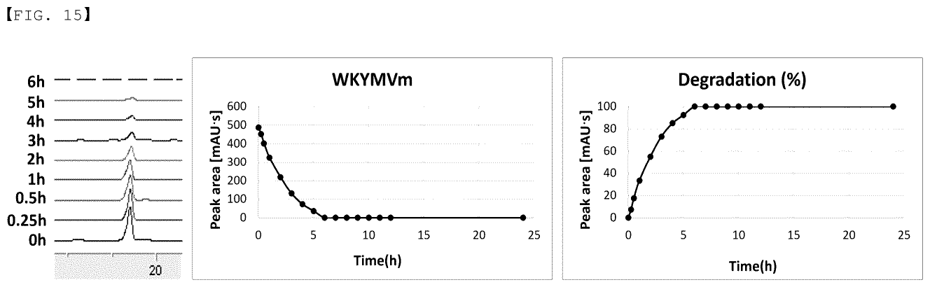

To measure the ex vivo stability of the eight analogs comprising a β³-amino acid residue, the degradation half-life of each analog was measured in a human liver S9 fraction. The degradation half-life was measured by culturing the peptide and serum together at 37° C. and measuring the degree to which the compound was reduced using HPLC at each hour. The WKYMVm peptide and its analogs all contain W and Y, so measurement was possible without interference from other compound signals by taking advantage of the fact that they exhibit strong absorption at 280 nm (FIG. 15).

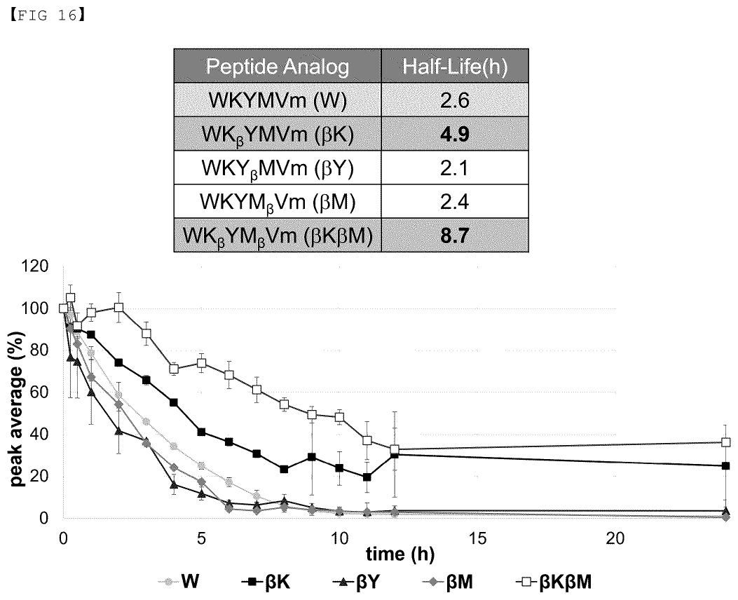

As a result, the WK_{β}YMVm peptide analog comprising one β³-amino acid residue had a half-life increased by about 1.9 times compared to the WKYMVm peptide, while the WKY_{β}MVm and WKYM_{β}Vm peptide analogs showed a half-life similar to that of the WKYMVm peptide (FIG. 16). In addition, the WK_{β}YM_{β}Vm peptide analog to which two β³-amino acid residues were introduced showed the highest stability with a half-life increased by about 3.3 times compared to the WKYMVm peptide (FIG. 16). The WK_{β}YM_{β}Vm peptide analog had a structure in which three non-natural residues out of six residues, comprising the C-terminal D-methionine, were arranged alternately with natural residues, and it showed results that were consistent with the prediction that it would exhibit the greatest stability against proteolytic enzymes. In addition, the WK_{β}Y_{β}M_{β}Vm peptide analog to which three non-natural residues were introduced showed high stability to the extent that no half-life was observed within the 24-hour measurement period.

5.3 Derivation of Optimized Sequences of WKYMVm Peptide Analogs to which a β-Amino Acid Residue was Introduced

As a result of summarizing the neutrophil mobility regulating effect and the half-life increasing effect of the peptide analogs to which a β³-amino acid residue was introduced, it was confirmed that the WK_{β}YM_{β}Vm and WKY_{β}YM_{β}Vm peptide analogs in which two residues were substituted with a non-natural residue can serve as effective substances for an FPR agonist therapeutic agent with improved ex vivo stability.

5.4. In Vitro Activity Results of WKYMVm Peptide Analogs to which a Peptoid Residue was Introduced

As a result of comparing and analyzing the effect of enhancing neutrophil mobility by the peptide analogs to which one peptoid residue was introduced, K peptoid, Y peptoid, and M peptoid were able to promote neutrophil mobility with similar activity to WKYMVm. Based on this, three additional peptide analogs to which two peptoid residues were introduced were synthesized.

5.5. Ex Vivo Stability Measurement Results of WKYMVm Peptide Analogs to which a Peptoid Residue was Introduced

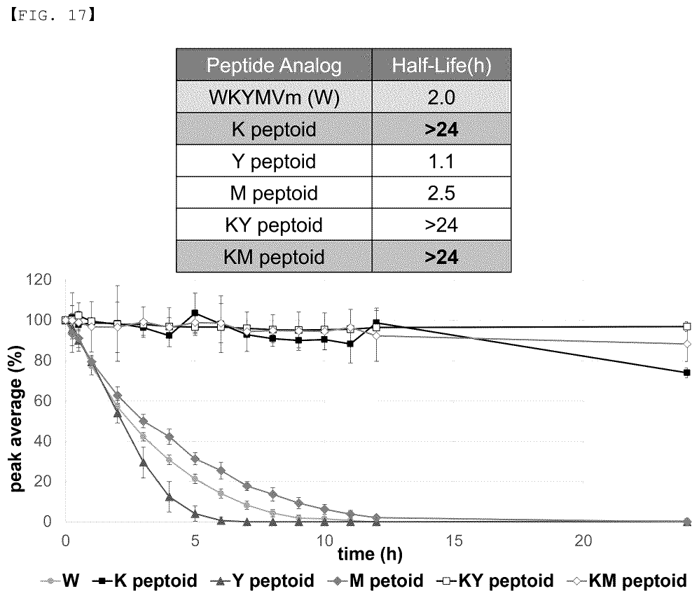

The ex vivo stability of the analogs to which a peptoid residue was introduced was measured using the same method as the stability measurement method for the analogs comprising a β-amino acid residue. As a result, among the peptide analogs comprising one peptoid residue, M peptoid and Y peptoid showed a half-life similar to or slightly shorter than that of the WKYMVm peptide (FIG. 17). On the other hand, it was confirmed that K peptoid, KY peptoid, and KM peptoid had very high stability so that more than half of them was not degraded even after 24 hours (FIG. 17).

The WKYMVm peptide analog of the present invention has effects of increasing stability by increasing in vivo degradation half-life and of promoting activation of innate immune responses by neutrophils through activation of formyl peptide receptors, and thus can be effectively used in preventing or treating a disease that is prevented or treated by formyl peptide receptors, especially an infectious disease or inflammatory disease.

The description of the present invention described above is for illustrative purposes, and those skilled in the art will understand that the present invention can be easily modified into other specific forms without changing the technical idea or essential features of the present invention. Therefore, the embodiments described above should be understood in all respects as illustrative and not restrictive. For example, each component described as a single form may be implemented in a distributed manner, and similarly, components described as distributed may also be implemented in a combined form.

The scope of the present invention is indicated by the claims described below, and all changed or modified forms derived from the meaning and scope of the claims and their equivalent concepts should be construed as being comprised in the scope of the present invention.

What is claimed is:

1. A WKYMVm peptide analog comprising an amino acid sequence of the following:
Trp (W)-Lys (K)-Tyr (Y)-Met (M)-Val (V)-D-Met (m),
wherein at least one amino acid residue selected from the group consisting of Lys (K), Tyr (Y), and Met (M) in the amino acid sequence is substituted with a β³-amino acid residue or a peptoid residue.

2. The WKYMVm peptide analog of claim 1, wherein one amino acid residue selected from the group consisting of Lys (K), Tyr (Y), and Met (M) in the amino acid sequence is substituted with a β³-amino acid residue or a peptoid residue.

3. The WKYMVm peptide analog of claim 1, wherein two amino acid residues selected from the group consisting of Lys (K), Tyr (Y), and Met (M) in the amino acid sequence are substituted with a β³-amino acid residue or a peptoid residue.

4. The WKYMVm peptide analog of claim 1, wherein amino acid residues of Lys (K), Tyr (Y), and Met (M) in the amino acid sequence are substituted with a β³-amino acid residue or a peptoid residue.

5. The WKYMVm peptide analog of claim 1, wherein the WKYMVm peptide analog in which at least one amino acid residue selected from the group consisting of Lys (K), Tyr (Y), and Met (M) in the amino acid sequence is substituted with a β³-amino acid residue has a following chemical formula:

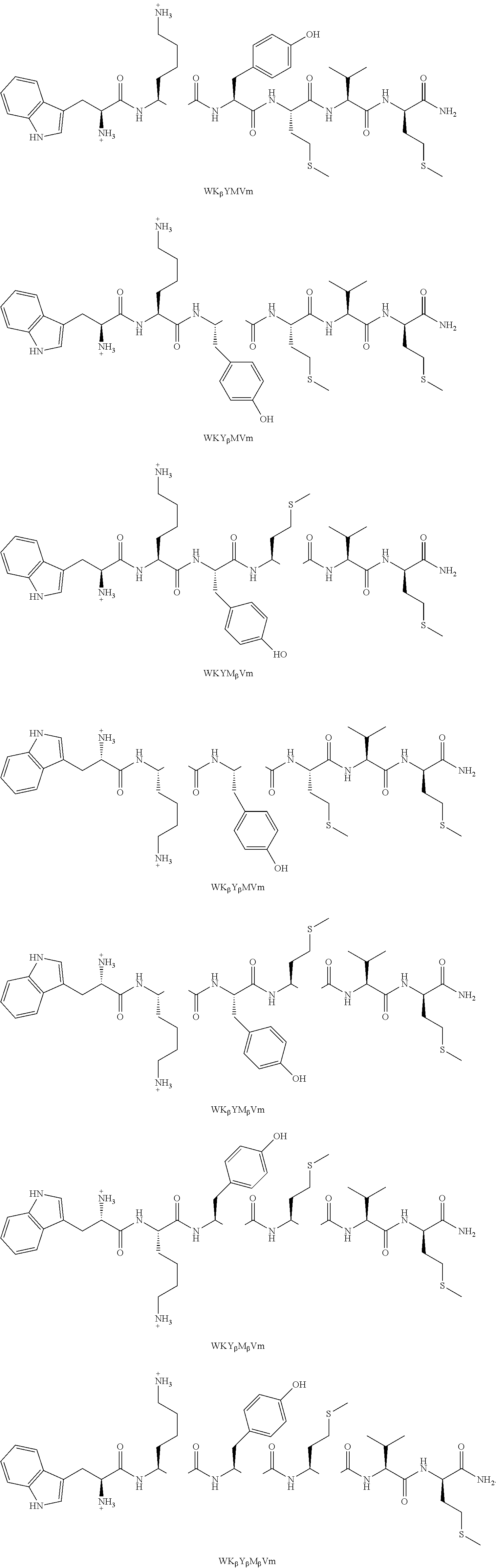

6. The WKYMVm peptide analog of claim 1, wherein the WKYMVm peptide analog in which at least one amino acid residue selected from the group consisting of Lys (K), Tyr (Y), and Met (M) in the amino acid sequence is substituted with a peptoid residue has a following chemical formula:

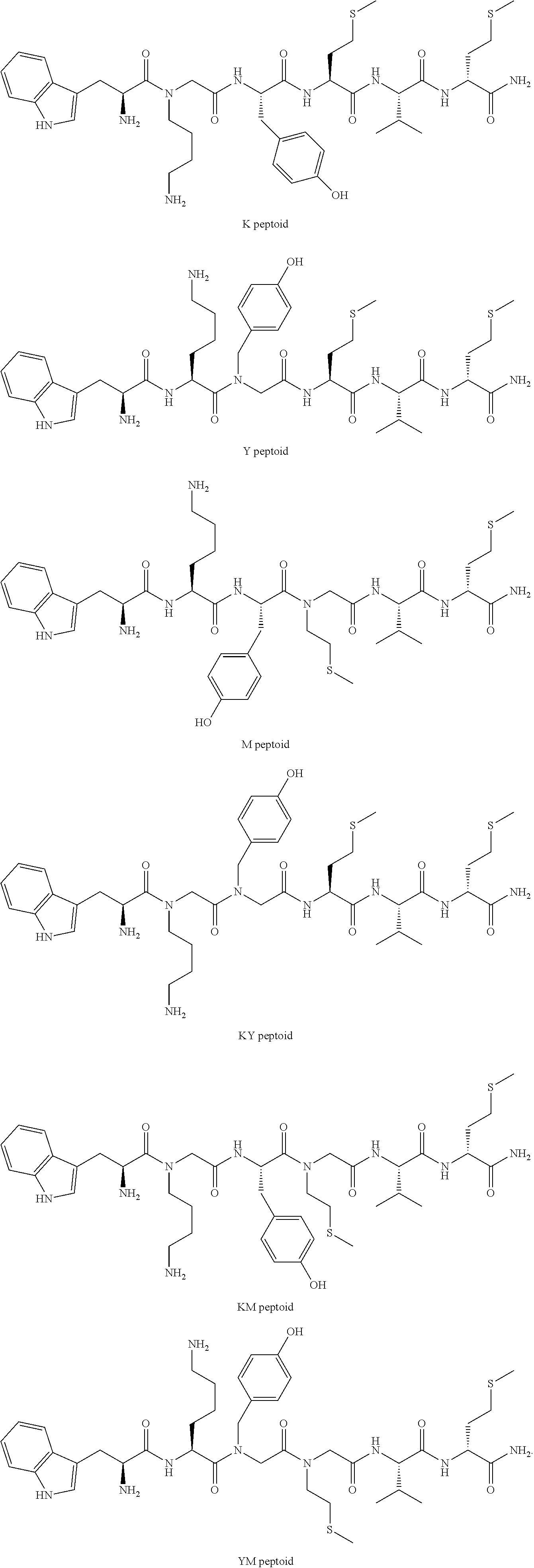

7. The WKYMVm peptide analog of claim 1, wherein the WKYMVm peptide analog induces or promotes activation of a formyl peptide receptor (FPR).

8. The WKYMVm peptide analog of claim 1, wherein the WKYMVm peptide analog has one or more characteristics selected from the group consisting of:
(i) promoting activation of innate immune responses by neutrophils;
(ii) inducing an increase in calcium ions in neutrophils;
(iii) promoting production of reactive oxygen species by neutrophils;
(iv) promoting degranulation activity of neutrophils;
(v) promoting mobility of neutrophils; and
(vi) regulating cytokine production in neutrophils.

9. The WKYMVm peptide analog of claim 1, wherein the WKYMVm peptide analog has an increased in vivo degradation half-life compared to a WKYMVm peptide.

10. A composition comprising a peptide analog of claim 1.

11. An immunity-enhancing method comprising administering the composition of claim 10 in a subject in need thereof.

12. A method of preventing or treating an infectious disease, comprising administering the composition of claim 10 in a subject in need thereof.

13. The method of claim 12, wherein the infectious disease is selected from the group consisting of respiratory syncytial (RS) virus infection, keratitis, conjunctivitis, tuberculosis, tuberculous osteomyelitis, tuberculous peritonitis, tuberculous meningitis, cervical lymphadenitis, infectious myositis, fungal infection, acute viral hepatitis, acute gastroenteritis, acute vaginitis, Kikuchi disease, norovirus enteritis, brain abscess, encephalitis, herpes simplex, herpes simplex encephalitis, shingles, Escherichia coli infection, dengue fever, rash, tinea capitis, legionellosis, leptospirosis, listeriosis, malaria, syphilis, melioidosis, viral meningitis, bacterial meningitis, botulism, brucellosis, Vibrio vulnificus sepsis, bacterial shigellosis, Pseudomonas infection, hand, foot-and-mouth disease, amoebic dysentery, lymphadenitis, cervicitis, enterohemorrhagic Escherichia coli infection, sepsis, pulmonary tuberculosis, and staphylococcal infection.

14. A method of preventing or treating an inflammatory disease, comprising administering the composition of claim 10 in a subject in need thereof.

15. The method of claim 14, wherein the inflammatory disease is selected from the group consisting of ankylosing spondylitis, psoriatic arthritis, osteoarthritis, rheumatoid arthritis, arthritis, dermatitis, atopic dermatitis, conjunctivitis, periodontitis, gingivitis, rhinitis, allergic rhinitis, chronic sinusitis, otitis media, pharyngitis, tonsillitis, bronchitis, pneumonia, emphysema, bronchial asthma, pulmonary fibrosis, gastric ulcer, gastritis, Crohn's disease, enteritis, colitis, hemorrhoids, urethritis, gout, rheumatic fever, lupus, fibromyalgia, tendinitis, tenosynovitis, peritendinitis, myositis, hepatitis, cystitis, nephritis, Sjogren's syndrome, multiple sclerosis, ulcers, and wounds.
